(19) United States
(12) Patent Application Publication
(10) Pub. No.: US 2013/0084271 A1
(43) Pub. Date: Apr. 4, 2013

(54) Probiotic composition for pets and method of providing the same

(71) Applicant: Kelly Foods Corporation, Berlin, MD (US)

(72) Inventors: Robert W. Kelly, Medina, OH (US); Gerhard Josef Poppel, Dagboro, DE (US)

(73) Assignee: Kelly Foods Corporation, Berlin, MD (US)

(21) Appl. No.: 13/573,705

(22) Filed: Oct. 3, 2012

Related U.S. Application Data
(60) Provisional application No. 61/542,557, filed on Oct. 3, 2011.

(51) Int. Cl. A23K 1/165; A61K 35/74; A61K 36/064; B05C 17/02; A61P 1/00; A23K 1/16; B65D 83/00; A61K 35/66; A61P 3/02

(52) U.S. Cl. 424/93.44; 424/93.1; 424/93.4; 424/93.45; 424/93.51; 426/61; 426/62; 222/251; 239/302; 426/56; 426/7; 401/209

(57) ABSTRACT

A probiotic composition, delivery device, method of forming, and a method for delivery are disclosed. The composition may be in the form of a spray, mist, film, or the like suitable for use with any pre-formulated pet food product, whether in solid or liquid form, and further to the use of the spray directly on the coat or skin of an animal. The probiotic spray may also be applied directly into a cavity of the animal, for example into its mouth or nasal passages. The probiotic composition provides multiple strains or species of viable bacteria that provide health benefits when ingested, and is shown herein primarily for consumption by or use on/in animals, though the invention is not limited in any way to use strictly for animal intake, but may also be suitably used for human ingestion.

This application claims the priority of U.S. Provisional Application Ser. No. 61/542,557, filed Oct. 3, 2011, the disclosure of which is incorporated herein by reference in its entirety.

### FIELD OF THE INVENTION

The invention relates to a probiotic composition and to a method for forming and delivery thereof. More specifically, the invention relates to a probiotic composition which serves as a probiotic food additive in the form of a spray or mist suitable for use with any pre-formulated pet food product, whether in solid or liquid form, and further to the use of the liquid probiotic composition in the form of a spray, mist or film directly on the coat or skin of an animal. The probiotic composition when in the form of a spray, mist, or film may also be applied directly into a cavity of the animal, for example into its mouth or nasal passages. The probiotic composition provides multiple strains or species of viable bacteria that provide health benefits when ingested, and is shown herein primarily for consumption by or use on/in animals, though the invention is not limited in any way to use strictly for animal intake, but may also be suitably used for human ingestion.

### BACKGROUND OF THE INVENTION

The invention relates to the application of a probiotic composition to food or other item to be ingested by a host. Further, the invention relates to the application of the probiotic composition contemplated herein directly onto or into the host, as on to the hosts skin or coat, or in a cavity of the host. As used herein, the term “host” may include any living species, whether animal or human, however, the exemplary probiotic composition is particularly suited to non-human animals (including birds). The term probiotic is generally understood to refer to a live microbial strain that when ingested contributes positive bacteria to the micro-flora of the gastrointestinal tract of the host. Probiotics are naturally-occurring bacteria, for example, beneficial lactic acid bacteria and other beneficial species including yeasts, which are present in the gastrointestinal tract of healthy hosts. These microorganisms participate in a variety of positive, health promoting activities in human and animal physiology by inhabiting the intestinal tract and contributing to good health by improving nutritional intake and protecting against disease. Some of the positive attributes of probiotic species, also referred to herein at times as “strains”, include enhanced digestive function, maintenance of microbial balance, protection against pathogenic bacteria, production of necessary vitamins and growth factors, and enhanced immune response capability of the host.

Dozens of microorganisms have been shown to have desirable probiotic qualities, at least in vitro. However, ingested bacteria are normally killed in the stomach before they have a chance to become established in the intestinal tract of the host. A probiotic species or strain may be any microorganism strain (such as lactobacilli, bifidobacteria, streptococci, sacharomycis, etc) shown in published research to have one or more of the following positive attributes: in vitro adherence to epithilial cells, in vitro antimicrobial activity, in vitro resistance to bile, hydrochloric acid, and pancreatic juice, anticarcinogenic activity (reduction of carcinogens) in clinical trials, immune modulation or stimulation in human clinical trials, reduction of intestinal permeability in human clinical trials, and colonization of the GI tract in human clinical trials.

A small number of strains have been shown to colonize the human gastrointestinal (GI) tract in clinical trials. Such strains or species may be referred to as “implantable strains”, meaning a microbial strain native to the human GI tract (i.e. lactobacilli or bifidobacteria, etc.) that has been shown to survive passage through the GI tract, i.e. it appears live in the stool, or to persist on biopsies of the GI mucosa after cessation of feeding. This is thought to be a necessary prerequisite for any health benefits to be conferred with regard to use in human hosts. A few strains have shown they both colonize the GI tract and confer specific benefits in human clinical trials. These are the most credible variety of probiotic, and while the list of such is growing, there still are only a small handful of probiotic species that meet the criteria, most of which are not available commercially in the US market. Of the foregoing, a few implantable strains have been shown to have one or more specific health benefits, and therefore have demonstrated clinical usefulness. Some examples of benefits that have been shown include reduced intestinal permeability (LGG), enhancement of immune function (various strains), and treatment of infection.

McFarland L V, Bemasconi P “Saccaromyces boulardii, a review of an innovative biotherapeutic agent” Microbial Ecology in Health and Disease, 6:1578-171 1993, provides a listing of known probiotic strains and some of the benefits thereof, with regard to use in human hosts, for treatment of disturbed microflora. Some of the known strains include: Saccaromyces boulardii, yeast available in capsule form; L. casei strain DN-114001, in yogurt form; L. reuteri, available in yogurt form; L. acidophilus NCFB 1748, available in milk; L. rhamnosus VTT E-97800, research strain; L. rhamnosus 271, available as “PrimaLiv” fermented oatmeal soup; L. casei strain Shirota, research strain; L. casei GG; Lactobacillus GG; and L. plantarum 299v, among others. It is noted that often a benefit seen with regard to human hosts is also seen in animal hosts, though not always.

Similarly, there are known and available pet foods that contain the foregoing strains, generally for the same purpose, i.e., to improve the performance of the digestive tract with regard to being able to accept and incorporate certain foods/vitamin and nutrient sources without difficulty or discomfort, as well as to reduce the incidence of intestinal tract-related or originating diseases or conditions. As such, it has been known to incorporate the probiotic directly into the pet food at the time of formulation. This, however, is not always satisfactory given that each individual animal may have a different level of need, depending on size, extent of disease, and other factors that can affect intestinal health, including over-all environment. Therefore, even though pet food may be formulated as per the size of pet it is intended to feed, i.e., small, medium or large breed dog, for example, this may still not be precise enough with regard to amount of the probiotic ingested to treat or prevent a condition.

There remains a need for a product suitable for use with any pre-formulated pet food product, whether solid or liquid, or for direct application to the skin or coat of an animal, or for application directly into a cavity of the animal. Further, there remains a need for such product to be provided in an easily administered form, suitable for use by the consumer public as well as professionals.

### BRIEF SUMMARY OF THE INVENTION

In one embodiment, the probiotic composition disclosed herein provides a pet food additive. More specifically, the invention relates to a probiotic pet food additive, in the form of a spray or mist suitable for use with any pre-formulated pet food product, whether in solid or liquid form. The probiotic composition provides multiple strains or species of viable bacteria that provide health benefits when ingested, and is shown herein primarily for consumption by or use on/in animals, though the invention is not limited in any way to use strictly for animal intake, but may also be suitably used for human ingestion.

In another embodiment, the probiotic composition is provided for delivery in the form of a spray or mist, directly on the coat or skin of an animal. The probiotic composition provides multiple strains or species of viable bacteria that provide health benefits when applied to an animal's skin or coat.

In still another embodiment, the probiotic composition is provided for delivery in the form of a spray or mist for application directly into a cavity of the animal, for example into its mouth or nasal passages. The probiotic composition provides multiple strains or species of viable bacteria that provide health benefits when applied directly to the lining of a cavity of the animal.

In yet another embodiment, a method is provided for delivery of the probiotic composition in the form of a spray or mist, suitable for application to pre-formulated pet food, on the skin or coat of the animal, or for direct delivery into a cavity of the animal.

In yet another embodiment, a method for dispensing a liquid composition comprising multiple probiotic species includes providing a dispensing device containing a liquid probiotic composition comprising multiple probiotic species and discharging the liquid probiotic composition from the dispensing device onto at least one of a pre-formulated wet or dry food source for intake by a host, onto the body of a host for ingestion by the host and/or to eliminate pathogens and promote healing, or onto litter used by the host.

In yet another embodiment a delivery device includes a probiotic composition comprising one or more probiotic species, in a liquid format and a dispenser which holds the probiotic composition, the dispenser including a delivery mechanism for delivering portions of the probiotic composition from the dispenser.

In another embodiment, a method for formulating a liquid probiotic composition that includes multiple probiotic species includes combining a palatability enhancing system with a fermentation broth which includes multiple strains of probiotic organisms and fermenting the broth to increase a concentration of at least some of the strains of probiotic organisms and form a liquid probiotic composition comprising palatability enhancing agent derived from the palatability enhancing system which renders the liquid probiotic composition palatable to at least one non-human animal.

### DETAILED DESCRIPTION OF THE INVENTION

The invention relates to a probiotic composition and to a method for delivery thereof. More specifically, in one embodiment, the invention relates to a probiotic food additive, in the form of a spray or mist suitable for use with any pre-formulated pet food product, whether in solid or liquid form. The probiotic composition provides multiple strains or species of viable bacteria that provide health benefits when ingested by a host. Though the probiotic composition is shown herein primarily for consumption by or use on/in animals, the invention is not limited in any way to such use. One skilled in the art will readily comprehend the application of the disclosed embodiments for use with a human host as well.

Probiotics have been known for use in preventing and treating gastrointestinal afflictions. Generally, the various strains known and proven useful are added to pet food at the time of manufacture, either as a component of the food, or, for example, as a coating on pet food. Further, they are commonly used either alone or with only one or two other such strains. Such use has proven beneficial, but results in the end user being limited to only those limited combinations provided in the available pet food products. Further, the end user is relegated to having to accept the food with the predetermined amount of the probiotic already included as part of the food.

There is currently no vehicle available to the public for providing a probiotic additive that includes multiple probiotic strains or species in order to address a multitude of potential gastrointestinal afflictions. The present probiotic composition may provide for example up to about 15 strains, e.g., 11 strains of probiotic microorganisms. It is commonly accepted, and scientifically supported, that the more strains employed and the higher the concentration of those strains per intake, the better the benefit received. As such, use of the probiotic composition in accord with the disclosure improves the intestinal health of the animal, which promotes the animals over-all health and well-being, thus providing for longevity and better quality of life. Another benefit of the probiotic composition provided herein is the capability for the user to more specifically tailor the amount of the additive used to fit the needs of an individual animal.

Some of the benefits commonly attributed to the use of probiotic strains include: increased natural resistance to infectious disease in the gastrointestinal tract and a first line of defense against disease; prevention of dangerous fungal overgrowth; reduction or elimination of some allergic reactions; antibiotic action against certain pathogenic bacteria; enhanced resistance against viruses; optimized digestive processes, allowing maximum nutritional benefit from food; improved resistance to toxic bowel problems; resistance to diarrhea; stimulation of the immune system; improved resistance to food poisoning; production of needed nutrients, such as vitamin K, one form of which the body itself cannot make; improved lactose intolerance conditions; reduction of cholesterol levels; antioxidants, which inhibit the destructive effects of oxidation; and improved resistance to stress of all kinds. As such, it is seen that the use of probiotics can contribute significantly to an overall improved quality of life and longevity for the animal.

The probiotic composition, in one embodiment, may include any combination of live, naturally-occurring probiotic strains, or microorganisms. For example, the combination of strains may be selected from a group including, lactic acid bacteria, such as Bifidobacteria species, e.g., Bifidobacterium animalis, Bifidobacterium bifidum, and Bifidobacterium longum, Enterococcus species, e.g., Enterococcus lactis, Enterococcus thermophilus, Lactobacillus species, e.g., Lactobacillus acidophilus, Lactobacillus bulgaricus, Lactobacillus caesei, Lactobacillus fermentum, and Lactobacillus plantarum, Lactococcus lactis, rhodopseudomonas palustris. In addition to one or more of these lactic acid bacteria, the probiotic strains may also include yeasts, such as sacharomyces cerevisiae, and other bacteria such as Bacillus subtilis, Streptococcus thermophiles, and other like strains of probiotic and/or yeast, and combinations thereof. Probiotic strains which are suited to specific animals may be employed. In one embodiment, the probiotic is used in the form of the liquid fermentation product thereof.

In one embodiment, the probiotic composition includes at least four, or at least five, or at least six probiotic species, e.g., selected from the above microorganism species. In one embodiment, all the above species are employed in forming the probiotic composition. In one embodiment, each of at least four probiotic species (e.g., from those above) is present in a concentration of at least 100 colony forming units (CFU) per milliliter (CFU/ml), or at least 200 CFU/ml of the probiotic composition that is used to treat the host or food product.

The probiotics combined for inclusion in the probiotic composition disclosed herein may further be combined with other ingredients to supplement the spray product. For example, the probiotic mixture may be combined with one or more of water, preferably purified, active dry yeast, cane molasses, calcium oxide, magnesium oxide, salt, palatability enhancing agents such as natural flavoring, and other known pet food additives. The majority of the probiotic composition may be water, such as at least 51 wt. %, or at least 60 wt. %, or at least 70 wt. %, or at least 80 wt. %, of the probiotic composition is water. In the spray product in accord herewith, the maximum moisture level may be, for example, 99%, e.g., 98.5%. For a more viscous liquid composition, such as that used in a roller ball applicator, one or more thickeners maybe incorporated into the probiotic composition. The total lactic acid bacterium present in the probiotic composition may be, for example, up to about 100 billion colony forming units (CFU) per milliliter, or up to about 1 billion CFU/ml, or up to about 3 billion CFU/ml. This formulation is stable at room temperature and does not need refrigeration. The total lactic acid bacterium present in the probiotic composition may be, for example, at least about 1,000, or at least about 5,000, or at least about 10,000, or at least about 50,000 CFU/ml, or at least 100,000 CFU/ml, or at least about 300,000 CFU/ml. In some embodiments, the total lactic acid bacterium present in the probiotic composition may be at least about 100,000 CFU/ml.

The probiotic composition disclosed herein may be provided in the form of a spray, mist, film, liquid droplets, stream, aerosol, or the like. The liquid may be delivered from a delivery device which includes a dispenser having an appropriate delivery/dispensing mechanism. A variety of delivery devices with appropriate delivery mechanisms are contemplated. In one embodiment, there is provided a method and a mechanism for the spray application of any liquid type probiotic in general, for example as a stream, drop, spray or mist and, more specifically, any type of liquid probiotic for application on food, in drinks, or in any form of oral intake associated with, for example spraying, pumping, squirting or other form of liquid application, for use in association with a host, and specifically in association with animals, in particular dogs, cats, horses and small pets, though it is understood that the spray may be used in association with other types of animal species hosts and for human hosts.

Further the probiotic may be administered in the form of a liquid, for example as a stream, drop, spray or mist onto the skin and/or coat of the animal, and into the mouth or other body cavities of animals/humans to prevent any disorders, bad odors or other ailments or enhance the overall wellbeing and other benefits scientifically and commonly associated with the use of probiotic. As such, the spray may be dispensed from any known type of spray, pump or misting device, the same not being a limiting feature of the invention. When applied to the body of the host animal, such as its skin or its coat, the animal ingests the probiotic composition during grooming and/or the probiotic helps to eliminate pathogens, promote healing, and eliminate bacteria which cause odor on the animal. The composition maybe applied to specific sites on the skin to improve healing of a wound.

In one embodiment, the delivery device includes a dispenser in the form of a container and the dispensing mechanism includes a captured ball in a flanged opening of the container. The container holds a multi-use supply of the probiotic composition. The liquid probiotic composition is delivered to the surface of the ball in the form of a liquid film when the ball is rotated with respect to the flanged opening. For example, when a pet licks the ball or when the ball is rolled over a surface of the pet, such as the tongue, oral cavity, skin, or fur of the pet, an amount of the probiotic composition is delivered from the dispenser onto the animal. The gap between the ball and the flanged opening can be selected to provide a measured amount of the probiotic composition per lick of the ball. An example of a suitable dispensing system of this type is described in U.S. Pub. No. 2012/0067290, published Mar. 22, 2012, the disclosure of which is incorporated by reference in its entirety. The formulations disclosed in that application may be supplemented by the probiotic strains disclosed herein to form the probiotic composition, or may be replaced with the exemplary probiotic composition.

In another embodiment, the probiotic spray may be used to treat cat litter or any litter box to prevent odor and the potential development of harmful bacteria, which may then transfer to a host. A delivery device having a spray pump or the like as the delivery mechanism may be used to supply a measured amount of the probiotic composition to the litter.

The liquid probiotic composition in accord with anyone or more embodiments allows for convenient, safe, and accurate dosage of a combination of probiotic strains or species to a host without the need to use complicated, costly and/or cumbersome, calibrated metering devices. The delivery device used, such as a spray device, may include any type of pump bottle or canister, of any size, or any type of triggered device, as well as any type of pressurized device or container. Any of the foregoing may have a spray nozzle or hose, or other type of spray head. The device may include a pre-calibrated dosing mechanism, or a dialable dosing mechanism, or any other type of mechanism for determining and dispensing a desired dosage. The dispenser or container size and shape may be such that any universal design up to a 10 gallon size may be used, as well as a smaller household size, for example about 3 oz to about 20 oz containers up to about 1-3 gallon size containers in some embodiments, the dispensing device may deliver at least 1000 CFU of lactic acid bacterium per application. For example, the probiotic composition may be formulated such that one or two squirts from a spray bottle or one or two licks from a roller ball applicator may provide a dosage of at least 1000 CFU of lactic acid bacterium, e.g., per eating event (meal).

As formulated, the probiotic composition may include at least about 1000 and in some embodiments, up to, and even in excess of, about 1 billion viable colony-forming friendly organisms per spray or squirt of the device used (each squirt may be from 0.1-1 ml in volume, e.g., up to 0.5 ml in volume). This concentration of the probiotic composition will provide an amount of healthy, viable probiotic such that former problems related to the probiotic strains being destroyed by the stomach acid of the host prior to reaching the gastrointestinal tract are overcome, and the strains succeed in not only surviving the acidic conditions of the stomach, but also in populating the gastrointestinal tract of the host. It is contemplated, and has been shown, that a 12 oz bottle, if used as directed, will provide up to 6 months-worth of treatment for a small dog, 3 months-worth of treatment for a midsize dog, and one month-worth of treatment for a large breed dog. In addition, because the probiotic strains are natural products, and are carefully selected to provide a desired result, there is no danger of overdosing, and there are no negative side effects.

In one embodiment a pet, such as a cat or dog, is treated with the probiotic composition at least once a day for a plurality of days, such as for a period of at least seven days or at least thirty days. For example, with each meal, one or two (or more) squirts of the probiotic composition are applied to the pet food such that the animal is dosed with at least 1000 CFU of lactic acid bacterium per meal. In the case of a dog, for example, the probiotic composition may be supplied in this way one, two or three times a day. Or, the pet is encouraged to lick a roller ball dispenser at least once a day over the course of a number of days. Or the probiotic composition assists in eliminating pathogens from the litter box.

As is known in the art, probiotic compositions can be prepared by mixing a starter culture with water and sufficient nutrients at a suitable temperature and pH to enable the culture to replicate and increase in CFU content over the course of a few days or weeks, depending on the temperature selected, in a process generally referred to as fermentation. Fermentation conditions may be optimized to suit the particular strains of probiotic organisms being cultured. Fermentation processes are described, for example, in US Pub. No. 20110189147, the disclosure of which is incorporated herein by reference. However, the exemplary method is not limited to any particular fermentation process. As an example, the probiotic starter culture may be combined with one or more of water, preferably purified, active dry yeast, cane molasses, calcium oxide, magnesium oxide, and salt as suitable nutrients for growth at the start of the fermentation process.

The palatability enhancing agents may be added to the products of fermentation. In another embodiment, the palatability enhancing agents may be added prior to the completion of the fermentation process, for example, at the start or during the fermentation process. In some embodiments, palatability enhancing agents may be formed or modified during fermentation to make the resulting probiotic composition

In one embodiment, a palatability system which results in the palatability enhancing agents in part through the fermentation process includes one or more animal derived materials, such as meat, one or more proteolytic enzymes, e.g., proteinases such as papain, and one or more amino acids, such as cysteine, glycine, methionine, or combination thereof. One or more sugars such as dextrose, glucose, xylose or other saccharides or disaccharides may also be employed. For example, a palatability system may be formed from one or more organ meats, such as like chicken liver, chicken intestines or other animal protein, which have been pre-treated with proteolytic enzymes such as papain, and mixed with amino acids and sugars and which may be held at 60-130° C. for 10-300 minutes prior to spray drying. One example palatability composition includes:

90-95% meat components,

0.1-5% papain,

1-5% amino acids such as cysteine, glycine, methionine, or combination thereof, and

1-5% sugars such as dextrose, glucose, xylose, hexose, or combination thereof.

This spray dried palatability system can be added either prior or during the fermentation process of the probiotic starter culture. It was discovered that by doing so the palatability effect was significantly improved as shown in feeding test results. Other example palatability systems/enhancers are disclosed, for example, in U.S. Pat. No. 6,660,319, the disclosure of which is incorporated herein by reference in its entirety.

The resulting dried palatability system may be added to the fermentation broth containing the probiotic starter culture prior to or during fermentation. Or it can be added to the completed fermented mixture as the palatability enhancing agent. It has been found that adding the palatability system before fermentation is complete, e.g., at the start, results in a probiotic composition which is more palpable than when it is added subsequently. The resulting mixture containing the probiotic strains and palatability enhancing agents can be filtered, diluted, additional ingredients added, and/or otherwise post-processed to form the exemplary probiotic composition.

The palatability enhancing agent may be present in sufficient amount to increase the preference of a group of animals for pre-formulated animal food when the liquid probiotic composition is added to the pre-formulated animal food, as compared to the pre-formulated animal food without the liquid probiotic composition. For example, the spray dried palatability system/enhancer may be added to the fermentation broth or to the resulting fermented product at a concentration of at least 0.001% by weight, such as at least 0.01% by weight, and up to 10 wt. %.

It has been shown that the spraying of the probiotic onto the food in combination with the palatability enhancer has a significant palatability advantage versus the probiotic without the palatability enhancer.

The following Examples provide data regarding feeding and palatability trials using the probiotic spray additive in accord herewith. In each trial, a known pet food was used both alone and with the probiotic additive in accord with the invention and the results compared. The data demonstrates that there was a significant improvement in palatability when the additive was sprayed on dry dog and cat food. It is noted that continued, regular use of the product, throughout every stage of an animal's life, will result in longevity and enhanced quality of life for the host, and enjoyment of the owner.

### Example 1

### Canine Palatability Results

The purpose of the study was to assess the palatability of dog food with and without the probiotic composition. A kennel facility registered with the USDA No. 23-R-0126 under the Animal Welfare Act had a 12-hour-light/12-hour-dark cycle. Temperature ranges were kept, as far as possible, within targeted conditions (from 50° to 85° F.) in accordance with the Animal Welfare Act. Cages and feed bowls were cleaned daily and sanitized in accordance with the Animal Welfare Act.

Tagged dogs were fed with dry dog food (Joy® Special) either as received or treated with the example probiotic composition (Joy® Special plus Bio Live Stock).

### Methods and Procedures

Liquid Bio Live Stock was prepared by adding a meat-derived palatability enhancing system, as described above, to a probiotic culture starter culture and fermenting the mixture to produce a fermentation broth containing about 11 strains of probiotic organisms to form a probiotic composition containing probiotic strains and palatability enhancing agents.

In a first test, to create the test article, 400 grams of standard diet (Joy Special) was sprayed with 5 squirts or 2.75 ml of the Bio Live Stock.

Over the course of the two day study, each animal was offered 5.5 ml of Bio Live Stock. The B diet was 400 grams of standard diet dry. Twenty male and female Beagles identified by ear tattoo and cage number were presented the test diets on an individual basis. Two stainless steel bowls, each containing approximately 400 grams of diet, were offered once daily for 2 days. Bowl placement was reversed daily and both bowls were presented for 30 minutes. If one diet was completely consumed prior to the end of the 30 minutes, both bowls were removed.

In a second test, to create the test article, 400 grams of standard diet (Joy Special) was sprayed with 10 squirts or 5.5 ml of Bio Live Stock. Over the course of the two day study, each animal was offered 11 ml of Bio Live Stock, i.e., double that of the first test. The B diet was 400 grams of standard diet dry.

Food consumption and first choice preference were recorded for each dog. The statistics applied were the Wilcoxon test to establish nonparametric ranking of observed consumption differences and an Analysis of Variance in consumption. Individual t-tests were performed to determine the consumption preference of each dog. A Chi Square test was performed to establish the significance in first choice preference.

### Results

1. First Test

Over a 2 day period, the total consumption for the 20 dogs on study was as follows:
4200 grams of Joy Special plus Bio Live Stock (69%)
1912 grams of Joy Special (31%)

Joy® Special plus Bio Live Stock was consumed 2.20 times more than Joy Special.

According to individual t-tests, the consumption preference was as follows:
3 dogs preferred Joy® Special plus Bio Live Stock (15.0%)
1 dog preferred Joy® Special (5.0%)
16 dogs had no preference (80.0%)

The total first choice preference was as follows:
Joy® Special plus Bio Live Stock was chosen first 36 times.
Joy® Special was chosen first 3 times.
Joy® Special plus Bio Live Stock was a first choice 12.00 times more than Joy Special.

Individual first choice preference was as follows:
17 dogs chose Joy® Special plus Bio Live Stock first (85%)
0 dogs chose Joy® Special first (0%)
3 dogs had no preference (15%)

Of the total food consumed, the average intake ratio of each diet is as follows:
70.0% Joy® Special plus Bio Live Stock
30.0% Joy® Special

Using a Wilcoxon signed rank test, the distribution of consumption of the two diets was found not to be similar. Using an Analysis of Variance, the calculated F value equals 12.98. The value of F from the Distribution Table is 4.00 at the 5% level of significance. Therefore, Joy Special plus Bio Live Stock and Joy Special are significantly different at this level. The value of F from the Distribution Table is 7.08 at the 1% level of significance. Therefore, Joy Special plus Bio Live Stock and Joy Special are significantly different at this level. Chi Square analysis found a statistically significant difference in first choice preference.

These results demonstrate the palatability of the probiotic composition when used on a commercial dog food.

2. Second Test

In the second test (where double the quantity of Bio Live Stock of the First test was offered), over a 2 day period, the total consumption for the 20 dogs on study was as follows:
5001 grams of Joy® Special Plus Bio Livestock (81%)
1210 grams of Joy® Special (19%)

These results indicate that Joy® Special Plus Bio Livestock was consumed 4.13 times more than Joy Special.

According to individual t-tests, the consumption preference was as follows:
5 dogs preferred Joy® Special Plus Bio Livestock (25.0%)
2 dogs preferred Joy® Special (10.0%)
13 dogs had no preference (65.0%)

The total first choice preference was as follows:
Joy® Special Plus Bio Livestock was chosen first 37 times
Joy® Special was chosen first 3 times

Joy® Special Plus Bio Livestock was a first choice 12.33 times more than Joy Special.

Individual first choice preference was as follows:
17 dogs chose Joy Special Plus Bio Livestock first (85%)
0 dogs chose Joy Special first (0%)
3 dogs had no preference (15%)

Of the total food consumed, the average intake ratio of each diet is as follows:
79.2% Joy Special Plus Bio Livestock
20.8% Joy Special

Using a Wilcoxon signed rank test, the distribution of consumption of the two diets was found not to be similar. Using an Analysis of Variance, the calculated F value equals 35.51. The value of F from the Distribution Table is 4.00 at the 5% level of significance. Therefore, Joy Special Plus Bio Livestock and Joy Special are significantly different at this level. The value of F from the Distribution Table is 7.08 at the 1% level of significance. Therefore, Joy Special Plus Bio Livestock and Joy Special are significantly different at this level. Chi Square analysis found a statistically significant difference in first choice preference.

### Example 2

### Feline Palatability Results

The purpose of the study was to assess the palatability of cat food with and without the probiotic composition. Cats were fed with dry cat food either as received (Purina Cat Chow) or treated with the example probiotic composition (Purina Cat Chow Special plus Bio Live Stock).

### Methods and Procedures

A cattery facility registered with the USDA No. 23-R-0126 under the Animal Welfare Act had a 12-hour-light/12-hour-dark cycle and temperature ranges were kept, as far as possible, within targeted conditions (from 50° to 85° F.) in accordance with the Animal Welfare Act. Cages, feed bowls, water bowls, and litter boxes were cleaned daily and sanitized in accordance with the Animal Welfare Act.

Twenty male and female cats identified by ear tattoo and cage number were presented the test diets on an individual basis. Two stainless steel bowls, each containing approximately 100 grams of diet, were offered once daily for 2 days. Test Article A contained 100 grams of Purina Cat Chow plus 6 squirts or 3 ml of Bio Live Stock. Over the course of the two day study, cats were offered 6 ml of Bio Live Stock. Diet B consisted of their standard diet. Bowl placement was reversed daily and both bowls were presented for 4 hours. If one diet was completely consumed prior to the end of the 4 hours, both bowls were removed. Food consumption and first choice preference were recorded for each cat.

### Results

Over a 2 day period, the total consumption for the 20 cats on study was as follows:
1477 grams of Purina Cat Chow plus Bio Live Stock (62%)
913 grams of Purina Cat Chow (38%)

Purina Cat Chow plus Bio Live Stock was consumed 1.62 times more than Purina Cat Chow.

According to individual t-tests, the consumption preference was as follows:
1 cat preferred Purina Cat Chow plus Bio Live Stock (5.0%)
1 cat preferred Purina Cat Chow (5.0%)
18 cats had no preference (90.0%)

The total first choice preference was as follows:
Purina Cat Chow plus Bio Live Stock was chosen first 25 times
Purina Cat Chow was chosen first 15 times

This indicates that Purina Cat Chow plus Bio Live Stock was a first choice 1.67 times more than Purina Cat Chow without the Bio Live Stock.

Individual first choice preference was as follows:
6 cats chose Purina Cat Chow plus Bio Live Stock first (30%)
1 cat chose Purina Cat Chow first (5%)
13 cats had no preference (65%)

Of the total food consumed, the average intake ratio of each diet is as follows:
61.2% Purina Cat Chow plus Bio Live Stock
38.8% Purina Cat Chow

Using a Wilcoxon signed rank test, the distribution of consumption of the two diets was found to be similar. Using an Analysis of Variance, the calculated F value equals 8.53. The value of F from the Distribution Table is 4.00 at the 5% level of significance. Therefore, Purina Cat Chow plus Bio Live Stock and Purina Cat Chow are significantly different at this level. The value of F from the Distribution Table is 7.08 at the 1% level of significance. Therefore, Purina Cat Chow plus Bio Live Stock and Purina Cat Chow are significantly different at this level. Chi Square analysis found no statistical significance in first choice preference.

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims.

1. A method for dispensing a liquid composition comprising multiple probiotic species comprising:
providing a dispensing device containing a liquid probiotic composition comprising multiple probiotic species; and
discharging the liquid probiotic composition from the dispensing device onto at least one of:
a pre-formulated wet or dry food source for intake by a host,
the body of a host, and
litter used by the host.

2. The method of claim 1, wherein the probiotic species include at least one lactic acid bacterium.

3. The method of claim 2, wherein the probiotic species include at least one of a bifidobacteria species and a lactobacillus species.

4. The method of claim 1, wherein the multiple probiotic species include probiotic species that are selected from the group consisting of Bifidobacterium animalis, Bifidobacterium bifidum, Bifidobacterium longum, Enterococcus lactis, Enterococcus thermophilus, Lactobacillus acidophilus, Lactobacillus bulgaricus, Lactobacillus caesei, Lactobacillus fermentum, Lactobacillus plantarum, Lactococcus lactis, rhodopseudomonas palustris, sacharomyces cerevisiae, and Streptococcus thermophiles.

5. The method of claim 1, wherein the multiple probiotic species include probiotic species include at least four of Bifidobacterium animalis, Bifidobacterium bifidum, Bifidobacterium longum, Enterococcus lactis, Enterococcus thermophilus, Lactobacillus acidophilus, Lactobacillus bulgaricus, Lactobacillus caesei, Lactobacillus fermentum, Lactobacillus plantarum, Lactococcus lactis, rhodopseudomonas palustris, sacharomyces cerevisiae, and Streptococcus thermophiles.

6. The method of claim 1, wherein the liquid composition contains a total of at least 1000 CFU/ml of lactic acid bacteria.

7. The method of claim 1, wherein the discharging of the liquid composition comprises discharging a portion of the liquid composition which portion provides the host with a dose of at least 1000 CFU of lactic acid bacteria per eating event.

8. The method of claim 7, wherein the discharging of the liquid composition is repeated at least daily for a plurality of days.

9. The method of claim 1, wherein the host is a non-human animal.

10. The method of claim 1, wherein the liquid probiotic composition comprises a palatability enhancing agent.

11. The method of claim 10, wherein the palatability enhancing agent is present in sufficient amount to increase the preference of a group of non-human animals for pre-formulated animal food when the liquid probiotic composition is added to the pre-formulated animal food, over pre-formulated animal food without the liquid probiotic composition.

12. The method of claim 10, wherein the palatability enhancing agent is the result of an enzymically treated meat product that is added during fermentation of the probiotic species.

13. The method of claim 9, wherein the total palatability enhancing agent is at least 0.001 wt. % of the liquid probiotic composition.

14. The method of claim 10, wherein the body of the host comprises at least one of the skin of the host, the coat of the host, a cavity of the host, and a tongue of the host.

15. A dispensing device comprising:
a probiotic composition comprising one or more probiotic species, in a liquid format; and
a dispenser which holds the probiotic composition, the dispenser including a delivery mechanism for delivering portions of the probiotic composition from the dispenser.

16. The dispensing device of claim 15, wherein the delivery device comprises at least one of a pump liquid dispenser, a spray liquid dispenser, a mist liquid dispenser, and a roller ball dispenser.

17. The dispensing device of claim 15, wherein the probiotic species include at least one lactic acid bacterium.

18. The dispensing device of claim 15, wherein the multiple probiotic species include probiotic species that are selected from the group consisting of Bifidobacterium animalis, Bifidobacterium bifidum, Bifidobacterium longum, Enterococcus lactis, Enterococcus thermophilus, Lactobacillus acidophilus, Lactobacillus bulgaricus, Lactobacillus caesei, Lactobacillus fermentum, Lactobacillus plantarum, Lactococcus lactis, rhodopseudomonas palustris, sacharomyces cerevisiae, and Streptococcus thermophiles.

19. The dispensing device of claim 15, wherein the liquid composition contains a total of at least 1000 CFU/ml of lactic acid bacteria.

20. The dispensing device of claim 15, wherein the delivery mechanism is configured for discharging a portion of the liquid composition which provides a dose of at least 1000 CFU/ml of lactic acid bacteria.

21. The dispensing device of claim 15, wherein the liquid probiotic composition comprises a palatability enhancing agent.

22. A method for formulating a liquid probiotic composition comprising multiple probiotic species comprising:
combining a palatability enhancing system with a fermentation broth which includes multiple strains of probiotic organisms;
fermenting the broth to increase a concentration of at least some of the strains of probiotic organisms and form a liquid probiotic composition comprising palatability enhancing agent derived from the palatability enhancing system which renders the liquid probiotic composition palatable to at least one non-human animal.

23. The method of claim 22, wherein the palatability enhancing system comprises an enzymically-treated meat product.

24. The method of claim 23, wherein the palatability enhancing system comprises at least one of:
at least one proteolytic enzyme; and
at least one amino acid.
